(19) United States
(12) Patent Application Publication
(10) Pub. No.: US 2021/0209095 A1
(43) Pub. Date: Jul. 8, 2021

(54) Apparatus and Method for Combining Free-Text and Extracted Numerical Data for Predictive Modeling with Explanations

(71) Applicant: Textician, LLC, Cambridge, MA (US)

(72) Inventor: Stephen I. Gallant, Cambridge, MA (US)

(21) Appl. No.: 17/142,330

(22) Filed: Jan. 6, 2021

Related U.S. Application Data
(60) Provisional application No. 62/957,629, filed on Jan. 6, 2020.

(51) Int. Cl. G06F 16/248; G16H 10/00; G06F 16/2457

(52) U.S. Cl. CPC G06F 16/248; G06F 16/24573; G16H 10/00

(57) ABSTRACT

Apparatus and method to combine unstructured free text with structured data to make predictive modeling easier and better. Structured data is received from applying an extractor to the unstructured free text or from a database query of a related database. This permits unstructured model-building to be used when data also comes from structured data, also facilitating explanations of inferences based upon both unstructured and structured key passages.

### CROSS-REFERENCE TO RELATED APPLICATIONS

The present application claims priority to U.S. Provisional Patent Application Ser. No. 62/957,629, entitled “Method for Combining Free-Text and Extracted Numerical Data for Predictive Modeling with Explanations” and filed Jan. 6, 2020, which is incorporated in its entirety herein by reference.

### TECHNICAL FIELD

The present invention relates to predictive modeling for decision making, and more particularly to predictive modeling based on free text.

### BACKGROUND ART

Free text, or simply text, is becoming ever more important because it serves as the basis for a growing number of applications, including finance-related prediction, article classification, search, direct marketing, and predictive analytics. Moreover, the amount of text being generated through social media postings is growing exponentially. Known inference engines build predictive models based on training data that consists of text and correct inferences about each text document. When an inference engine processes a new text document, the engine determines inferences based on the previously processed training data.

Important applications of text include classification of text by establishing a fixed set of classes and predicting membership of the text in at least one of the fixed set of classes; clustering documents into sets of documents that are similar to each other, but less similar to documents in other clusters; summarization to shorten text; and extraction of names, amounts, prices, etc. These operations make it easier to select only documents having certain labels of interest, or belonging to certain classes of interest, to shorten documents, and to find particular facts within documents.

Prevailing methods of combining unstructured free text and structured data begin with building both a structured predictive model and an unstructured predictive model. Yet there is much effort building structured predictive models that involves: data selection, requiring domain experts; finding corresponding fields in a database, requiring database experts; rolling up data, for example combining dozens of temperature readings into a summary statistic; and fixing missing values for fields. Therefore, an apparatus and method that can greatly simplify processing and ease the task of providing explanations for inferences would be beneficial.

### SUMMARY OF THE EMBODIMENTS

In accordance with one embodiment of the invention, an apparatus for building predictive models has at least one processor and a memory including instructions that, when executed by the at least one processor, cause the at least one processor to receive first structured data and generate, from the first structured data, a first set of data words, wherein each data word in the first set of data words has a prefix and a value. The instructions also cause the at least one processor to combine the first set of data words to form first combined text. The instructions further cause the at least one processor, using a predictive modeling engine for free text, to analyze the first combined text and build a predictive model from the first combined text.

In a related embodiment, the memory further includes instructions that, when executed by the at least one processor, cause the at least one processor to receive first free text, wherein the first structured data corresponds to data in the first free text and wherein to combine the first set of data words includes combining the first free text and the first set of data words to form the first combined text.

Alternatively or in addition, the memory further includes instructions that, when executed by the at least one processor, cause the at least one processor to generate the first structured data from the first free text.

In a further related embodiment, the first structured data includes at least one datum and to generate the first set of data words includes, for each datum of the at least one datum: generating a prefix from at least one of a name and a description of the datum determining if a value of the datum is a numerical value; if the value is not a numerical value, appending the value to the prefix to form a data word of the first set of data words; if the value is a numerical value, generating at least one description of the value, and appending each of the at least one description to the prefix to form at least one data word of the first set of data words.

Alternatively or in addition, generating the at least one description of the value includes calculating a mean and a standard deviation of a set of values having the same prefix and the at least one description of the value is based on the value, the mean, and the standard deviation.

The first structured data may be received from a database. The first combined text may include medical data, and the predictive model built by the predictive modeling engine, when executed, may predict a set of medical codes. The set of medical codes may be one of a set of ICD-10 codes and a set of CPT codes.

In accordance with a related embodiment of the invention, an apparatus for classifying data using the predictive model built by the predictive modeling engine, wherein the predictive model predicts membership in a set of classes, includes at least one processor and a memory, the memory including instructions that, when executed by the at least one processor, cause the at least one processor to receive second structured data and generate, from the second structured data, a second set of data words, wherein each data word in the second set of data words has a prefix and a value. The instructions further cause the at least one processor to combine the second set of data words to form second combined text, wherein the second combined text is free text, and to execute the predictive model to classify the second combined text into at least one class of the set of classes.

Alternatively or in addition, each data word in the first set of data words is followed by a separator and the classifying includes generating an explanation for the classification made by the predictive model. The explanation for the classification may include a subset of the first set of data words.

In accordance with a further related embodiment of the invention, an apparatus for classifying free text using the predictive model built by the predictive modeling engine, wherein the predictive model predicts membership in a set of classes, includes at least one processor and includes a memory, the memory including instructions that, when executed by the at least one processor, cause the at least one processor to receive second free text and second structured data, wherein the second structured data corresponds to data in the second free text. The instructions further cause the at least one processor to generate, from the second structured data, a second set of data words, wherein each data word in the second set of data words has a prefix and a value. The instructions further cause the at least one processor to combine the second set of data words into second combined text, wherein the second combined text is free text, and to execute the predictive model to classify the second combined text into one of the set of classes.

Alternatively or in addition, the memory further includes instructions that, when executed by the at least one processor, cause the at least one processor to generate the second structured data from the second free text.

In a related embodiment, each data word in the second set of data words is followed by a separator and the classifying includes generating an explanation for the classification made by the predictive model. The explanation for the classification may include a subset of the second set of data words.

In accordance with another embodiment of the invention, a computer-implemented method of building predictive models includes receiving, by at least one processor, first structured data. The method also includes generating, by the at least one processor, from the first structured data, a first set of data words, wherein each data word in the first set of data words has a prefix and a value. The method further includes combining, by the at least one processor, the first set of data words to form first combined text; and using a predictive modeling engine for free text, running on the at least one processor, to analyze the first combined text and build a predictive model from the first combined text.

Alternatively or in addition, the method further includes receiving, by the at least one processor, first free text, wherein the first structured data corresponds to data in the first free text; and wherein combining the first set of data words includes combining the first free text and the first set of data words to form the first combined text.

In a related embodiment, the at least one processor generates the first structured data from the first free text. Alternatively or in addition, the at least one processor receives the first structured data from a database.

Alternatively or in addition, the first structured data includes at least one datum, and generating the first set of data words includes, for each datum of the at least one datum: generating a prefix from at least one of a name and a description of the datum; determining if a value of the datum is a numerical value; if the value is not a numerical value, appending the value to the prefix to form a data word of the first set of data words; if the value is a numerical value, generating at least one description of the value, and appending each of the at least one description to the prefix to form at least one data word of the first set of data words.

Alternatively or in addition, generating the at least one description of the value includes calculating a mean and a standard deviation of a set of values having the same prefix, and the at least one description of the value is based on the value, the mean, and the standard deviation.

In a related embodiment, the first combined text includes medical data, and the predictive model built by the predictive modeling engine, when executed, predicts a set of medical codes. The set of medical codes may be one of a set of ICD-10 codes and a set of CPT codes.

In a related embodiment, a computer-implemented method for classifying data using the predictive model, wherein the predictive model predicts membership in a set of classes, includes receiving, by at least one processor, second structured data and generating, by the at least one processor from the second structure data, a second set of data words, wherein each data word in the second set of data words has a prefix and a value. The method further includes combining, by the at least one processor, the second set of data words to form second combined text, wherein the second combined text is free text, and executing, by the at least one processor, the predictive model to classify the second combined text into at least one class of the set of classes.

Alternatively or in addition, each data word of the first set of data words is followed by a separator and the classifying includes generating, by the at least one processor, an explanation for the classification made by the predictive model. The explanation for the classification may include a subset of the first set of data words.

In a related embodiment, a computer-implemented method for classifying free text using the predictive model includes receiving, by at least one processor, second free text and second structured data, wherein the second structured data corresponds to data in the second free text; generating, by the at least one processor, from the second structured data, a second set of data words, wherein each data word in the second set of data words has a prefix and a value; combining, by the at least one processor, the second set of data words into second combined text, wherein the second combined text is free text; and executing, by the at least one processor, the predictive model to classify the second combined text into at least one class of the set of classes.

In a related embodiment, the at least one processor generates the second structured data from the second free text. Alternatively or in addition, each word of the second set of data words is followed by a separator and the classifying includes generating, by the at least one processor, an explanation for the classification made by the predictive model. The explanation for the classification may include a subset of the second set of data words.

### BRIEF DESCRIPTION OF THE DRAWINGS

The foregoing features of embodiments will be more readily understood by reference to the following detailed description, taken with reference to the accompanying drawings, in which:

FIG. 1 shows an apparatus for building predictive models in accordance with an embodiment of the present invention;

FIG. 2 depicts an apparatus for executing predictive models in accordance with an embodiment of the present invention;

FIG. 3 depicts a flowchart of a method for building predictive models in accordance with an embodiment of the present invention;

FIG. 4 shows a flowchart of a method for building predictive models in accordance with an embodiment of the present invention;

FIG. 5 shows a flowchart of a method for executing predictive models in accordance with an embodiment of the present invention;

FIG. 6 shows a flowchart of a method for executing predictive models in accordance with an embodiment of the present invention;

FIG. 7 depicts a flowchart for generating data words in accordance with an embodiment of the present invention;

FIG. 8 depicts a flowchart for generating data words in accordance with an embodiment of the present invention;

FIG. 9 depicts a flowchart for generating data words in accordance with an embodiment of the present invention;

FIG. 10 shows exemplary free text;

FIG. 11 shows exemplary structured text; and

FIG. 12 depicts exemplary data words.

### DETAILED DESCRIPTION OF SPECIFIC EMBODIMENTS

Definitions. As used in this description and the accompanying claims, the following terms shall have the meanings indicated, unless the context otherwise requires:

A “set” has at least one member.

“Free text” refers to unstructured text including, but not limited to, medical notes, articles, press releases, email, web pages, blogs, text messages, and tweets.

A “predictive model” is a statistical model built from known data that, when applied to new data, predicts membership of the new data in a set of specified classes. The predictive model may, for example, be based on at least one of logistic regression, decision trees, neural networks, analysis of variance (ANOVA), random forests, linear regression (ordinary least squares), ridge regression, time series analysis, generalized linear models, Bayesian analysis, and multivariate adaptive regression splines. In particular, a predictive model for text has inputs consisting of free text rather than numeric values, where said text may be internally represented by a list of numbers, by using methods such as Salton's vector space model, Deerwester's Latent Semantic Indexing, Gallant's Matrix Binding of Additive Terms, and other methods known to those skilled in the art.

A “predictive modeling engine” is an engine that builds predictive models. More specifically, a predictive modeling engine for text is an engine that builds predictive models using free text by applying text modeling and classification techniques to free text with associated class labels to produce a predictive model. Illustratively, the predictive modeling engine may use statistical methods and representations as exemplified in the preceding paragraph to analyze free text with associated class labels (i.e., correct inferences) and create a predictive model based on detected patterns in the free text.

An “explanation” for an inference of a text predictive model is a portion of the text that corresponds to said inference and thereby increases confidence in the correctness of said inference.

A “data word” is a set of characters, possibly including numerals and special characters such as underscores, that does not contain spaces or other word separating characters.

A “sentence separator” or “separator” is a set of characters that marks the end of a sentence, such as a period or a period following by one or more blank characters (“.”).

“Medical data” is data collected and/or generated in the medical field, including, but not limited to, notes from a medical professional, test results, vital parameter measurements, imaging data, medical histories, medical reports, diagnoses.

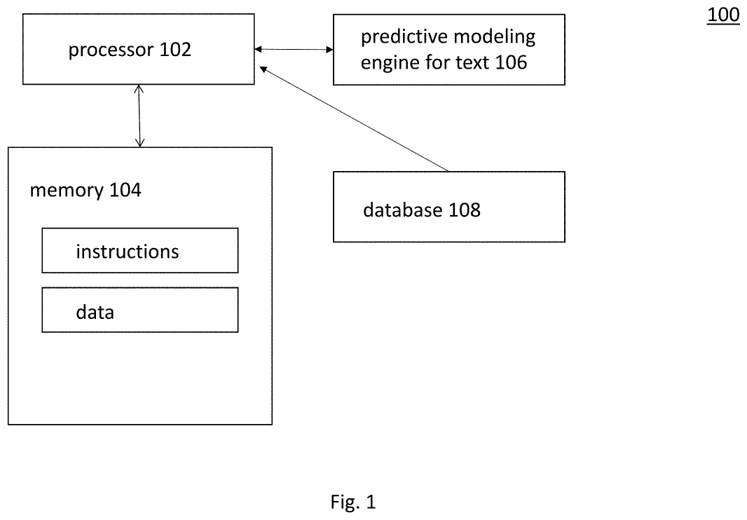

FIG. 1 shows an exemplary apparatus for building predictive models in accordance with an embodiment of the present invention. Apparatus 100 includes a processor 102 coupled to a memory 104. The memory 104 stores instructions for creating data words and/or combined text that can be executed by the processor 102, and the memory 104 stores data. While only embodiments having one processor are shown and described, it is expressly contemplated that two or more processors are used. The apparatus 100 further includes a predictive modeling engine 106. The predictive modeling engine 106 is coupled to the processor 102 and, through the processor, to the memory 104. Alternatively, the predictive modeling engine may be stored in the memory 104. In addition, a database 108 may be coupled to the processor 102.

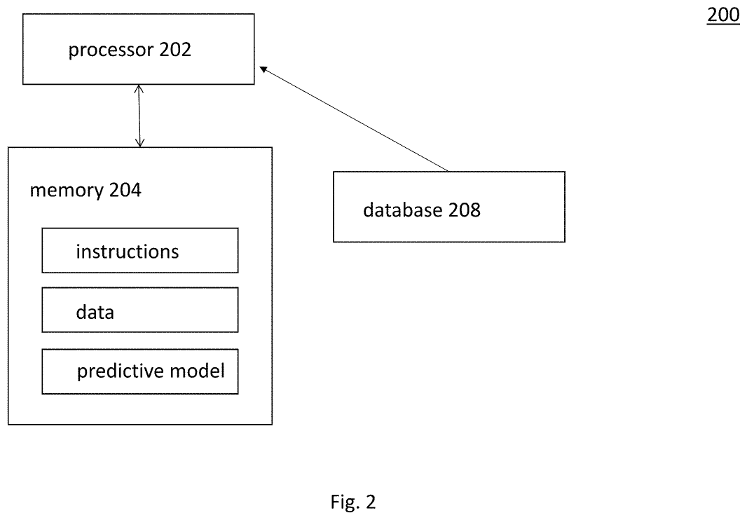

FIG. 2 shows an exemplary apparatus for executing predictive models in accordance with an embodiment of the present invention. Apparatus 200 includes a processor 202 coupled to a memory 204. The memory 204 stores instructions for creating data words and/or combined text that can be executed by the processor 202, and the memory 204 stores data. The memory 204 also stores a predictive model and instructions for applying the predictive model that can be executed by the processor 202. The predictive model may, for example, have been created by the predictive modeling engine 106 of apparatus 100. While only one processor is shown, it is expressly contemplated that two or more processors are used. The apparatus 200 may further include a database 208 coupled to the processor 202.

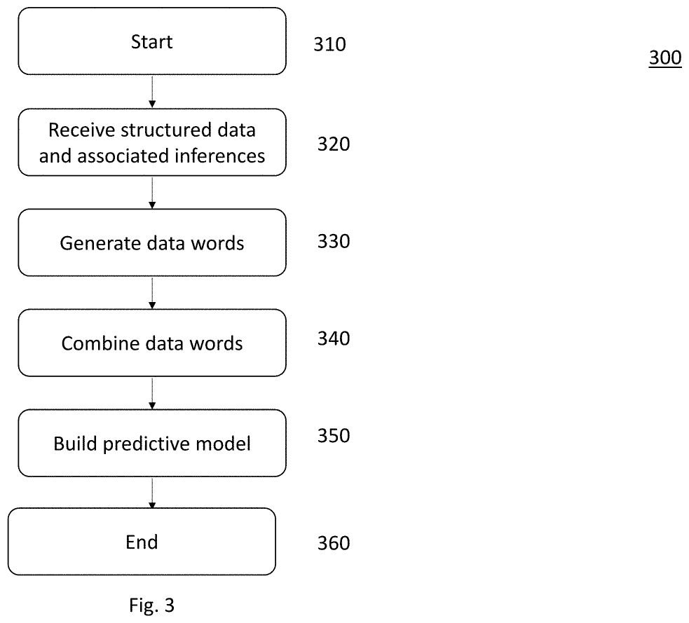

FIG. 3 depicts a flowchart of a method for building predictive models in accordance with an embodiment of the present invention. The method starts at step 310 and then proceeds to step 320 where structured data and associated training inferences are received. Illustratively, the structured data may be received by processor 102. The structured data may be received by the processor from a database, such as database 108. The database may have database fields and values associated with the fields, but not all database fields may be of interest. For example, only the field “patient temperature” may be of interest. A database query may be used to produce such fields of interest from the database. Some or all values may be absent for each selected field from the database.

Associated with the data in step 320 are desired inferences, i.e. classifications, that the predictive modeling engine uses to build a predictive model. For example, with structured data related to a patient's hospital stay, the classifications may be those medical codes associated with this hospital stay. A predictive model built by the predictive modeling engine can then predict these medical codes for similar hospital stays of other patients. However, it is expressly contemplated that no desired inferences are associated with the structured data, and no desired inferences are received by the processor 102. In such an alternative embodiment, the predictive modeling engine may employ unsupervised learning to build a predictive model.

In step 330, in accordance with embodiments of the present invention, the processor generates what the inventor calls data words. A data word is a single textual word characterized by having a prefix and a value, and each data word of a set of data words is optionally followed by a sentence separator. In addition, no spaces or blank characters are used in a data word, making a data word a single-word sentence when followed by an optional separator. The use of no spaces allows the text modeling methods to better build predictive models. The use of the separator allows for each data word to be read as a sentence by the predictive modeling engine and/or the predictive model. Although the separator is not needed for building and applying predictive models, it is used in the preferred embodiment for generating explanations for inferences.

Exemplarily, a sentence separator is a period followed by at least one space (“.”). However, any other string may be used in place of a period and spaces, for example an exclamation point. Moreover, alternative embodiments may employ sentence separators only following groups of data words having the same prefix parts, rather than following every data word. Additionally, if sentence explanations are not being used, the sentence separators after data words may be eliminated entirely. For example, data words may be followed just by a single blank character which normally separates text words.

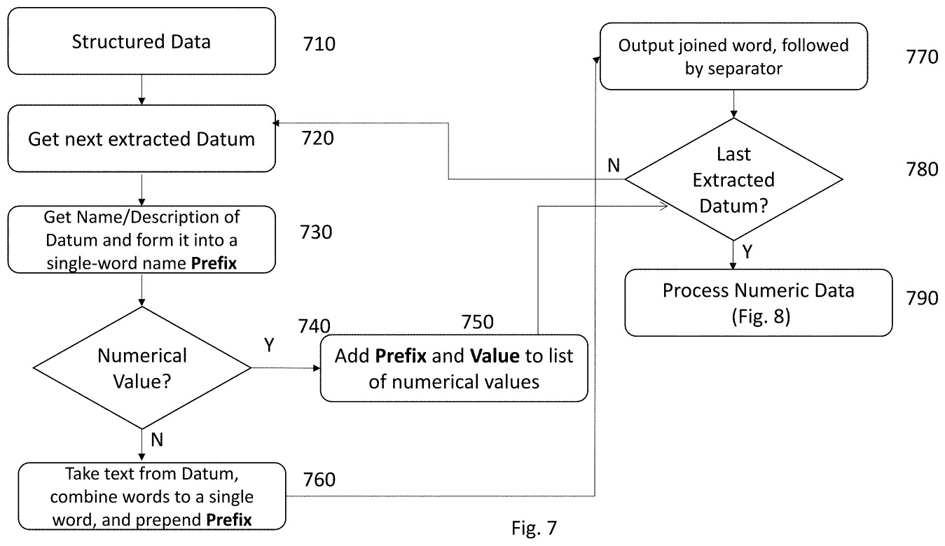

The set of data words may be generated from the structured data. Each piece of structured data may result in one or more data words. Exemplarily and as described in detail below with reference to FIG. 7, a data word can be generated from a single piece of structured data, a datum. The prefix of the data word may describe what type of data it is, e.g. “body_temperature_.” The value may be a textual value taken from the structured data, such as “rapidly_rising.” The resulting data word then is “body_temperature_rapidly_rising.” If the value is a numerical value, the piece of structured data may be converted to more than one data word. The relative size of the numerical value can be represented by one or more additional data words that represent where the numerical value stands relative to other values in the dataset that have the same prefix. For example, the datum “Temperature=103.4” may be converted to two data words: “body_temperature_high_among_noted_vals” and “body_temperature_very_high_among_noted_vals.” The generation of data words is described in detail below with reference to FIGS. 7, 8, and 9.

The method then proceeds to step 340, where the processor combines the generated data words to form combined text. Combining the data words may, for example, include appending the data words of the set of data words to one another.

In step 350, a predictive modeling engine for text, such as predictive modeling engine 106, is executed on the processor to build a predictive model from the combined text and its associated classifications. The predictive modeling engine may apply text modeling and classification techniques to the combined text to produce a predictive model. The predictive modeling engine predicts membership of the combined text in a set of specified classes. Exemplarily, the set of specified classes may be specified along with groupings of the structured data, or it may be determined in a different manner. For example, with a specific patient's hospital stay, the structured data for the stay may be accompanied by medical code classifications associated with said hospital stay. An example of a classification, to be used in medical coding, would be: “Patient should be assigned medical code E10.618.” The medical code may be selected from a set of medical codes, for example, from a selected revision of the International Statistical Classification of Diseases and Related Health Problems (ICD) such as ICD-10, or it may be selected from another set of medical codes such as Current Procedural Terminology (CPT). As another example, if the combined text corresponds to medical patient records, an exemplary class/classification may be: “Patient will be diagnosed with severe sepsis in the next 48 hours.” In another embodiment where the predictive model predicts financial markets, a further example of a classification may be: “Stock expected to drop by >=10% in the next day,”

Examples of existing text modeling and classification software are contained in easily obtained libraries (sklearn) for the Python programming language including tf-idf (TfidfVectorizer) and LSI/SVD, so-called “deep learning” techniques, and numerous other techniques known to those skilled in the art. It is important that the modeling software accept new words as regular words in its processing, because each data word of the set of data words will be, in general, a new word. The method ends at step 360.

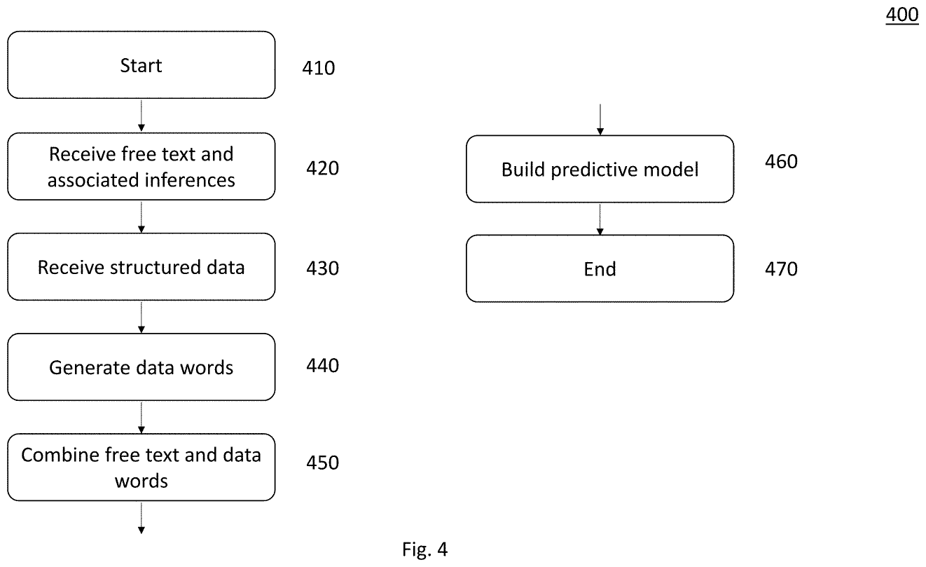

FIG. 4 depicts a flowchart of a method for building predictive models in accordance with an alternative embodiment of the present invention. The method starts at step 410 and then proceeds to step 420 where free text is received. Illustratively, the free text may be received by a processor such as processor 102. The free text may be a single document or it may be a collection of documents. The free text may be divided into groups wherein each group may be accompanied by desired inferences (classifications) that pertain to that group. For example, when a predictive model to predict medical codes from text is built, a group may consist of hospital progress notes and lab reports for a patient's visit to a hospital, and the classifications might consist of medical codes associated with said texts. It is, however, expressly contemplated that no desired inferences accompany each group, and no desired inferences are received by the processor 102, such as when the predictive modeling engine used is based on unsupervised learning.

In step 430, the processor then receives structured data that corresponds to the free text. The structured data may be received by the at least one processor from a database, such as database 108. The structured data may also be generated from the free text. To this end, an extractor may be applied to each document of free text to produce structured data for items of interest. For example, items of interest may be disease diagnoses, symptoms such as fever, measurements of numerical physical signs such as “Temperature=102.4,” and drugs administered. Different applications outside the medical field may extract different types of items of interest from the free text, such as people, places, stock prices, analyst reactions, and many others. Any of a number of known extractors may be employed, such as Comprehend by Amazon (Seattle, Wash.), cTAKES by the Apache Software Foundation (Forest Hill, Md.), and many others.

In step 440, the processor generates a set of data words from the structured data. Each data word of the set of data words has a prefix and a value, and each data word of the set of data words is optionally followed by a separator. Each piece of structured data may result in one or more data words. The generation of data words is described in detail below with reference to FIG. 7. Alternatively, if sentence explanations are not being used, the sentence separators after data words may be eliminated entirely.

The method then proceeds to step 450, where the processor combines generated free text and any free text to form combined text. Combining the free text and the data words may, for example, include appending the data words of the set of data words to the free text. Alternatively, the data words may be placed before the free text or may be interspersed with it. Moreover, the combined text may include only the data words.

In step 460, a predictive modeling engine, such as predictive modeling engine 106, is executed on the processor to build a predictive model from the combined text accompanied by associated classifications. The predictive modeling engine may apply text modeling and classification techniques to the combined text to produce a predictive model having a set of classes as described above. The method ends at step 470.

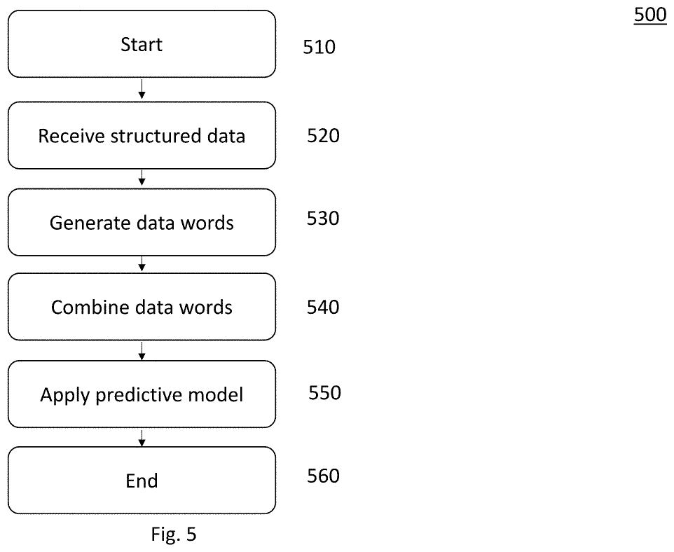

FIG. 5 shows a flowchart of a method for executing predictive models in accordance with an embodiment of the present invention. The method starts at step 510 and then proceeds to step 520 where data, such as structured data, is received. The data may, for example, be received by a processor such as processor 202. In step 530, the processor generates a set of data words from the structured data as described above with reference to FIG. 4 and as described below in more detail with reference to FIG. 7. In step 540, the data words are combined to result in combined text. The combined text is free text.

The method then proceeds to step 550. The processor executes software that applies a predictive model that is stored, for example, in memory 204 to classify the combined text. Examples of such software include Scikit-learn, a free software machine learning library for the Python programming language. Illustratively, the predictive model was built by predictive modeling engine 106. As described above, the predictive model may establish a set of classes and may predict membership of the combined text in at least one of the set of classes. For example, the predictive model may predict that the data is a member of the class “Patient will be diagnosed with severe sepsis in the next 48 hours.” Alternatively, the predictive model may predict that the combined text is a member of more than one class. For example, in an embodiment where the predictive model predicts financial markets, the combined text may be predicted to be a member of the class “Stock expected to drop by >=10% in the next day” and also a member of the class “Stock expected to drop by >=20% in the next week.”

The text modeling and classification software may additionally provide an explanation for the resulting classifications by identifying key sentences (or words or phrases) rated most highly by the model for any classification it predicts. By generating data words as sentences, this permits the explanation to be sentences from the original text and/or data words. One method for generating such sentences that are explanatory for a classification inference is to apply the predictive model to each sentence separately in the combined text, and to note those sentences with highest predictive score for said inference. Such a sentence with the highest predictive score may include one or more data words. The method ends at step 560.

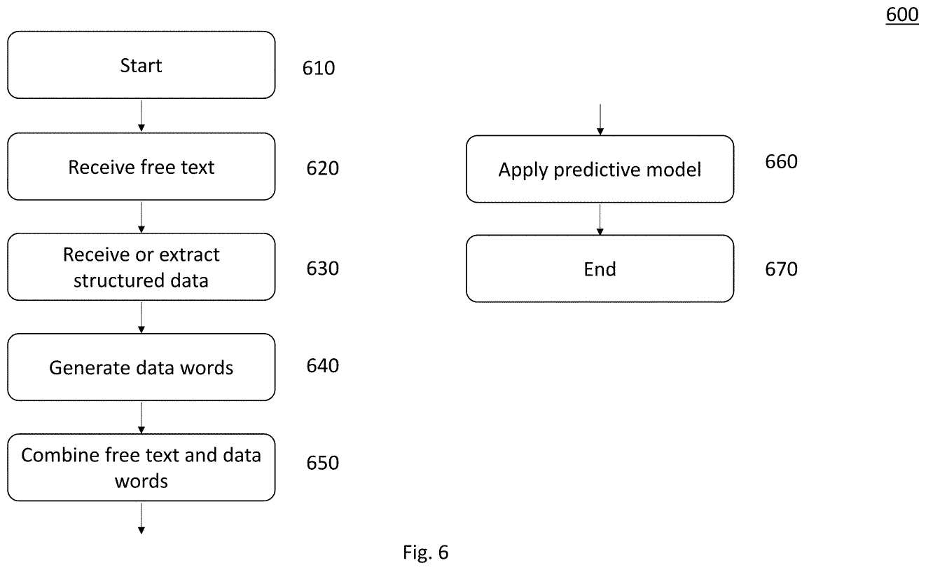

FIG. 6 depicts a flowchart of a method for executing predictive models in accordance with an alternative embodiment of the present invention. The method starts at step 610 and then proceeds to step 620 where free text is received. The free text may, for example, be received by a processor such as processor 202.

The method then proceeds to step 630. The processor receives structured data that corresponds to the free text. The structured data may be received from a database 208 or it may be generated or extracted from the free text as described above.

In step 640, the processor generates a set of data words from the structured data as described above with reference to FIG. 4 and as described below in more detail with reference to FIG. 7. In step 650, the data words and any free text are combined to result in combined text. Alternatively, the combined data may include the generated data words only.

The method then proceeds to step 660. The processor executes suitable software as illustrated above, to apply a predictive model that is stored in memory 204 to classify the combined text. Illustratively, the predictive model was built by predictive modeling engine 106. As described above, the predictive model may establish a set of classes and may predict membership of the combined text in at least one of the set of classes. Similar to what is described above with reference to FIG. 5, the predictive model may additionally generate explanations for its classifications. The method ends at step 670.

FIG. 7 depicts a flowchart of a method for generating data words in accordance with an embodiment of the present invention. Specifically, FIG. 7 shows how structured data is converted to data words.

The method starts at step 710 with the structured data received from a database or generated from a document or a set of documents. In step 720, the first or next datum of the structured data is considered.

In step 730, a single-word prefix for the current datum is determined. The prefix may be indicative of what type of data the datum is. For example, if the datum is one of several test results that are to be kept distinct, the prefix may be “GBS_test_” to name the “GBS” test. Similarly, the “RR” test result may receive the prefix “RR_test_”. Prefix names are the collections of structured data that are to be grouped. For example, if the method is designed to keep weights for males and for females differently, the prefixes “Weight_male” and “Weight_female_” may be used. Similarly, negated values could be separated by including “NEGATED_” in the prefix.

Exemplarily, underscores “_” are used to connect words in the prefix, but it is expressly contemplated to connect words in the prefix in other ways, such as CamelCode (“findingTestRR”). Blanks in the prefix are avoided so that it can be joined with a value to become a single data word.

The method then proceeds to step 740 where a decision is made if the value for the current datum is a number. If the value is a number, the prefix and the value are added in step 750 to a list of all such pairs, the list of numeric items. If the datum's value is not a number, in step 760 a single data word is created by combining the words in the value to a single word and prepending the prefix. For example, if body temperature is said to be rapidly rising, the prefix may be “body_temperature_”, the value may be “rapidly_rising”, and the resulting data word may be “body_temperature_rapidly_rising”.

The method creates such single-word data words to take advantage of text modeling software that permits new words and allows these new words to be an explanation for classification. Examples for text modeling software that permits new words are contained in easily obtained libraries (sklearn) for the Python programming language including tf-idf (TfidfVectorizer) and LSI/SVD, so-called “deep learning” techniques, and numerous other techniques known to those skilled in the art. Single-word sentences, consisting of data words with separators, can then be identified by the predictive model as an explanation for its classification predictions. Using data words as the explanation instead of free text enhances its usefulness. In step 770, a separator such as “.” may be added to the end of every data word. However, if only predictions are of interest and not the explanations for said predictions, then the separator may be omitted.

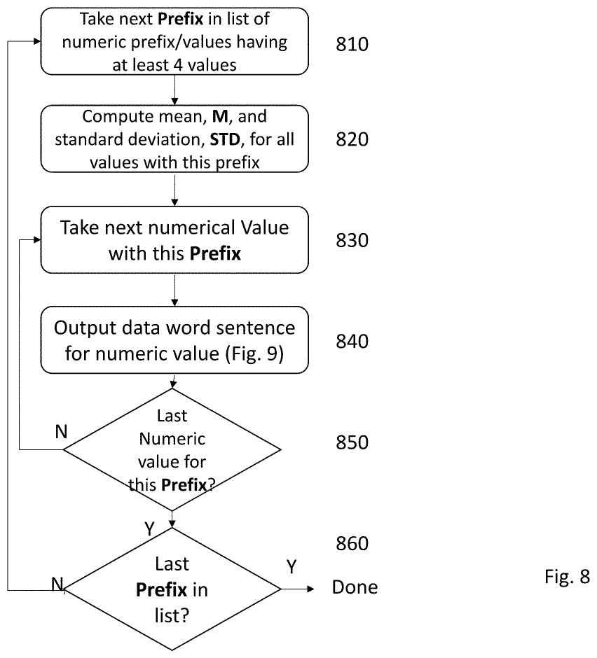

In step 780 it is determined if the current datum is the last datum of the structured data. If it is not the last datum, the method continues with step 720. If it is the last datum, the method proceeds to step 790 to process the numeric data as shown in detail in FIG. 8.

FIG. 8 depicts a flowchart of a method for generating data words in accordance with an embodiment of the present invention. Specifically, FIG. 8 shows how the list of numeric items is processed. In step 810, the next prefix having at least four values associated with it is selected. If there are no more such remaining prefixes, the method ends. The limit four is only exemplary and can be any fixed integer greater than zero.

In step 820 the mean M and standard deviation STD are computed for all values associated with the current prefix. If there are insufficient values to compute the STD, the STD may, for example, not be computed or may be set to M. Alternatively, the prefix and its associated values may be discarded from the list of numeric items.

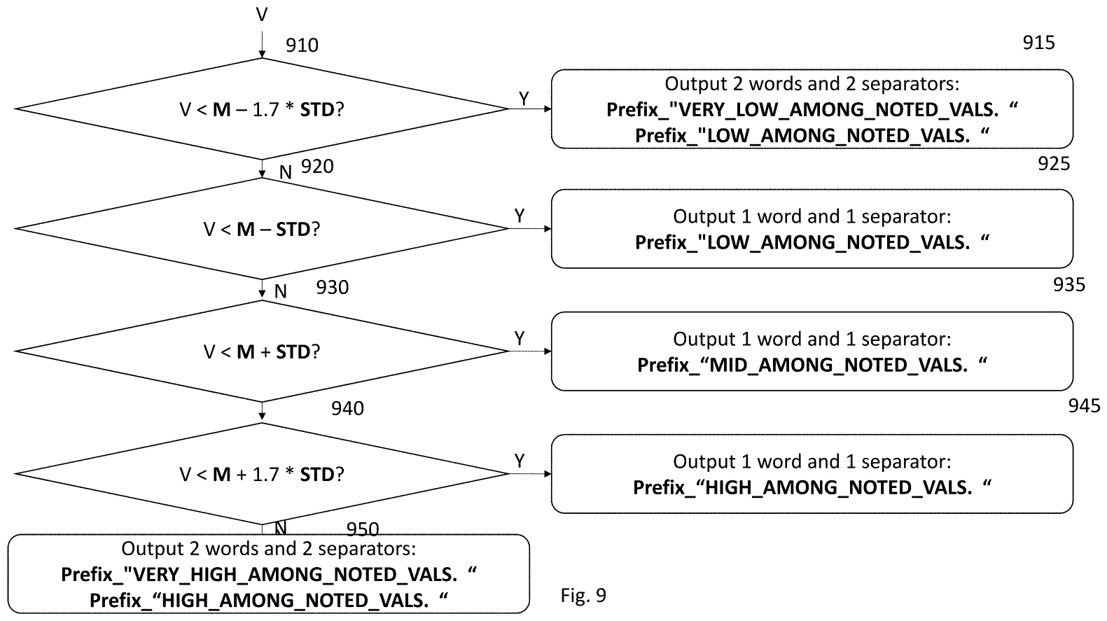

In step 830, the next numeric value with the current prefix is selected. A corresponding data word is generated in step 840. How the data word containing relative information for a numeric value is generated is described below with reference to FIG. 9.

The method then proceeds to step 850 to determine if the current value is the last numeric value for the current prefix. If there are more values left to process, the method returns to step 830. Otherwise, the method proceeds to step 860.

In step 860, it is determined whether the current prefix is the last prefix in the list of numeric items. If there are more prefixes to process, the method returns to step 810. Otherwise, the method ends.

FIG. 9 depicts a flowchart for generating data words in accordance with an embodiment of the present invention. Specifically, FIG. 9 shows how data words that include relative information for a numeric value are generated from a tuple of a value (V), a mean (M), and a standard deviation (STD), as generated by the method shown in FIG. 8. This is advantageous, because the production of relative information adds power to the predictive modeling process. For example, for modeling and prediction it is preferable to have “temperature 98.61” result in the same data word as “temperature 98.62”. The enhanced/structured combined text should therefore improve classification.

The method begins at step 910 and checks if V is a very low value compared to other values for this prefix. Illustratively, this decision may be made by determining if V is less than M−1.7*STD. If so, in step 915 two data words are generated. The first data word prepends the prefix to the string “VERY_LOW_AMONG_NOTED_VALS.”, resulting in a single word followed by an optional separator such as a period and a space. The second data word prepends the prefix to the string “LOW_AMONG_NOTED_VALS.”, also resulting in a single word followed by an optional separator. While the character strings are shown with uppercase characters here, it is not necessary that they only include uppercase characters. They may include only lowercase characters, or they may include a mix of uppercase and lowercase characters.

The reason for using two data words is to make it easier for machine learning techniques to handle cases that can either have low or very low values for a class the predictive modeling engine is learning to recognize. Also, separators are optionally added so that machine learning techniques can produce a data word as an explanation (basis) for an inference, if such explanations are desired. It is, however, expressly contemplated that separators are omitted.

If V is not a very low value, the method proceeds to step 920 to check if V is a low value compared to other values for this prefix. Illustratively, this decision may be made by determining if V is less than M−STD. If so, in step 925 a data word is generated that prepends the prefix to the string “LOW_AMONG_NOTED_VALS.”, resulting in a single word followed by an optional separator.

If V is not a low value, the method proceeds to step 930 to check if V is a mid-range value compared to other values for this prefix. Illustratively, this decision may be made by determining if V is less than M+STD. If so, in step 935 a data word is generated that prepends the prefix to the string “MID_AMONG_NOTED_VALS.”, resulting in a single word followed by an optional separator.

If V is not a mid-range value, the method proceeds to step 940 to check if V is a high value compared to other values for this prefix. Illustratively, this decision may be made by determining if V is less than M+1.7*STD. If so, in step 945 a data word is generated that prepends the prefix to the string “HIGH_AMONG_NOTED_VALS.”, resulting in a single word followed by an optional separator.

If V is not a high value, the method proceeds to step 950 and generates two data words. The first data word prepends the prefix to the string “VERY_HIGH_AMONG”NOTED_VALS.”, resulting in a single word followed by an optional separator. The second data word prepends the prefix to the string “HIGH_AMONG_NOTED_VALS.”, also resulting in a single word followed by an optional separator.

It is expressly noted that the above thresholds are exemplary only. Those skilled in the art may decide to use other thresholds for the decisions, for example “V<2*STD” in step 910. Also, they may decide to use three groupings rather than five, or any other number. Finally, the thresholds may be individually specified for the decisions. For example, if the prefix involves a body temperature, the lowest group may be set to “V<95 degrees”, regardless of M and STD for body temperature values. It also contemplated that a value may not have an STD associated with it because the STD was not computed. In that case, the method may, for example, output a data word that prepends the prefix to the string “MID_AMONG_NOTED_VALS.”, resulting in a single word followed by an optional separator.

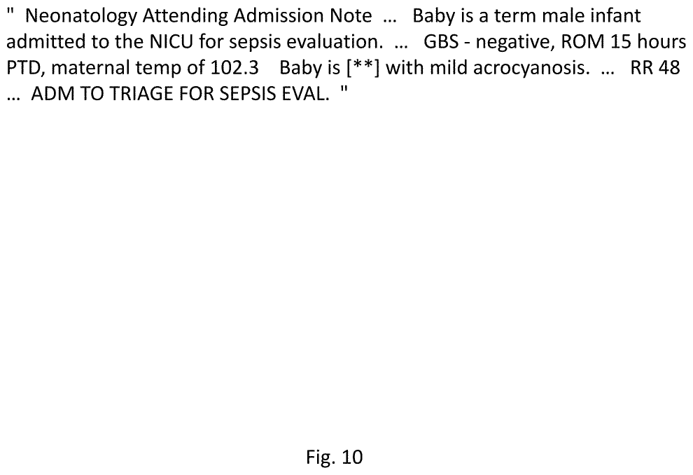

FIG. 10 shows exemplary free text. This example is from a medical note, but such free text could also be from a news report, web page, analyst recommendation, news release, or any of a multiplicity of other sources.

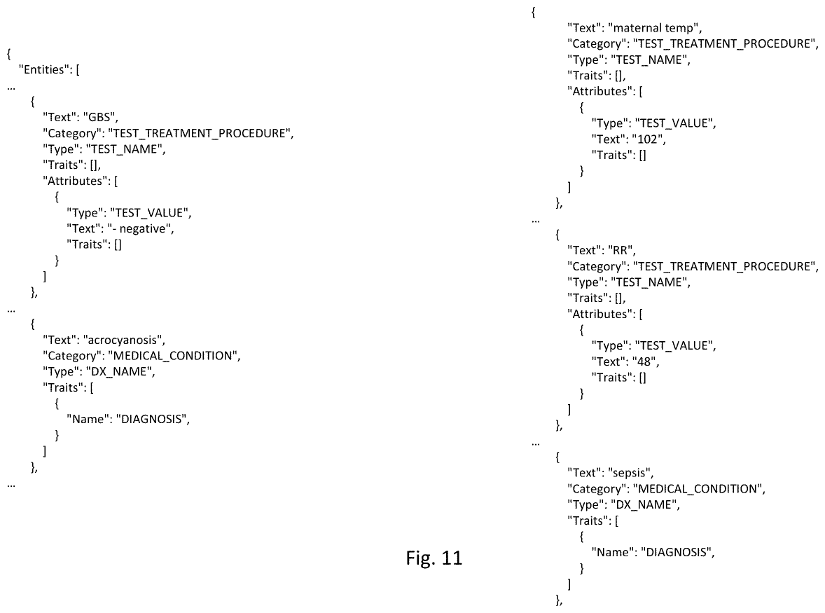

FIG. 11 shows exemplary structured text produced by an extractor. In this example, the extractor is Amazon's Comprehend. Here, the structured data is in JavaScript Object Notation (JSON) format. Instead, the output might be from a different extractor, such as the public domain software cTAKES, or it may be expressed in a different format for structured data, such as a variant of the Extensible Markup Language (XML). Alternatively, the structured data may be the result of a database query into a medical or other database.

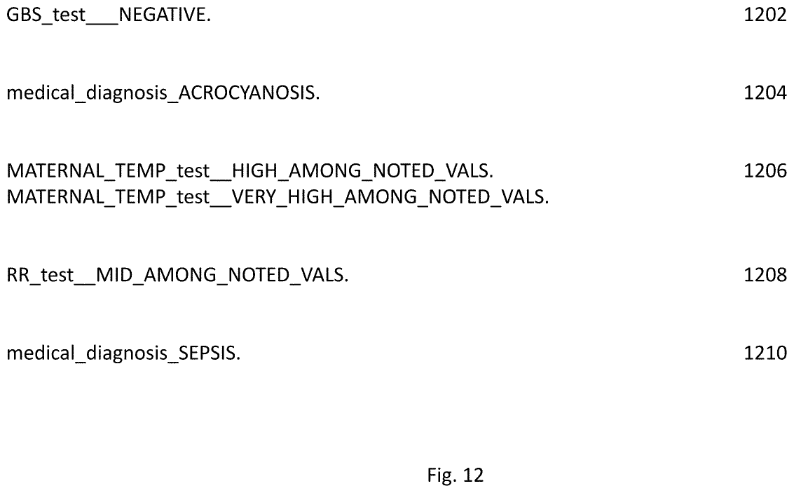

FIG. 12 shows an exemplary set of data words corresponding to the structured data shown in FIG. 11. Item 1202 and 1204 illustrate how multiple words or parts of words are joined by “_” into a single word. In item 1202, the prefix “GBS_TEST_” and the value “NEGATIVE”, plus an optional separator, are joined into the single word “GBS_TEST_NEGATIVE.”. The same method of combining is applicable to values. Items 1206, 1208, and 1210 show how numeric values are converted to ranges, as shown above in detail with reference to FIG. 9.

Certain embodiments described herein may be implemented as a computer program product for use with a computer system. Such implementations may include a series of computer instructions fixed either on a tangible medium, which is preferably non-transient and substantially immutable, such as a computer readable medium (e.g., a diskette, CD-ROM, ROM, flash drive, or fixed disk) or transmittable to a computer system, via a modem or other interface device, such as a communications adapter connected to a network over a medium. The medium may be either a tangible medium (e.g., optical or analog communications lines) or a medium implemented with wireless techniques (e.g., microwave, infrared or other transmission techniques). The series of computer instructions embodies all or part of the functionality previously described herein with respect to the system. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). Of course, some embodiments of the invention may be implemented as a combination of both software (e.g., a computer program product) and hardware. Still other embodiments of the invention are implemented as entirely hardware, or entirely software (e.g., a computer program product).

The embodiments of the invention described above are intended to be merely exemplary; numerous variations and modifications will be apparent to those skilled in the art. All such variations and modifications are intended to be within the scope of the present invention as defined in any appended claims.

What is claimed is:

1. An apparatus for building predictive models, the apparatus comprising:
at least one processor;
a memory, the memory including instructions that, when executed by the at least one processor, cause the at least one processor to:
receive first structured data;
generate, from the first structured data, a first set of data words, wherein each data word in the first set of data words has a prefix and a value;
combine the first set of data words to form first combined text; and
using a predictive modeling engine for free text, analyze the first combined text and build a predictive model from the first combined text.

2. The apparatus according to claim 1, wherein the memory further includes instructions that, when executed by the at least one processor, cause the at least one processor to receive first free text, wherein the first structured data corresponds to data in the first free text; and
wherein to combine the first set of data words includes combining the first free text and the first set of data words to form the first combined text.

3. The apparatus according to claim 2, wherein the memory further includes instructions that, when executed by the at least one processor, cause the at least one processor to generate the first structured data from the first free text.

4. The apparatus according to claim 1, wherein the first structured data includes at least one datum and wherein to generate the first set of data words comprises, for each datum of the at least one datum:
generating a prefix from at least one of a name and a description of the datum;
determining if a value of the datum is a numerical value;
if the value is not a numerical value, appending the value to the prefix to form a data word of the first set of data words;
if the value is a numerical value, generating at least one description of the value, and appending each of the at least one description to the prefix to form at least one data word of the first set of data words.

5. An apparatus for classifying data using the predictive model built by the apparatus of claim 1, wherein the predictive model predicts membership in a set of classes, the apparatus comprising:
at least one processor;
a memory, the memory including instructions that, when executed by the at least one processor, cause the at least one processor to:
receive second structured data;
generate, from the second structured data, a second set of data words, wherein each data word in the second set of data words has a prefix and a value;
combine the second set of data words to form second combined text, wherein the second combined text is free text; and
execute the predictive model to classify the second combined text into at least one class of the set of classes.

6. The apparatus according to claim 5, wherein each data word in the first set of data words is followed by a separator and wherein the classifying comprises generating an explanation for the classification made by the predictive model.

7. An apparatus for classifying free text using the predictive model built by the apparatus of claim 2, wherein the predictive model predicts membership in a set of classes, the apparatus comprising:
at least one processor;
a memory, the memory including instructions that, when executed by the at least one processor, cause the at least one processor to:
receive second free text and second structured data, wherein the second structured data corresponds to data in the second free text;
generate, from the second structured data, a second set of data words, wherein each data word in the second set of data words has a prefix and a value;
combine the second set of data words into second combined text, wherein the second combined text is free text; and
execute the predictive model to classify the second combined text into one of the set of classes.

8. The apparatus of claim 7, wherein the memory further includes instructions that, when executed by the at least one processor, cause the at least one processor to generate the second structured data from the second free text.

9. The apparatus according to claim 7, wherein each data word in the second set of data words is followed by a separator and wherein the classifying comprises generating an explanation for the classification made by the predictive model.

10. A computer-implemented method of building predictive models, the method comprising:
receiving, by at least one processor, first structured data;
generating, by the at least one processor, from the first structured data, a first set of data words, wherein each data word in the first set of data words has a prefix and a value;
combining, by the at least one processor, the first set of data words to form first combined text; and
using a predictive modeling engine for free text, running on the at least one processor, to analyze the first combined text and build a predictive model from the first combined text.

11. The method according to claim 10, the method further comprising:
receiving, by the at least one processor, first free text, wherein the first structured data corresponds to data in the first free text; and
wherein combining the first set of data words includes combining the first free text and the first set of data words to form the first combined text.

12. The method of claim 11, wherein the at least one processor generates the first structured data from the first free text.

13. The method of claim 10, wherein the first structured data includes at least one datum and wherein generating the first set of data words comprises, for each datum of the at least one datum:
generating a prefix from at least one of a name and a description of the datum;
determining if a value of the datum is a numerical value;
if the value is not a numerical value, appending the value to the prefix to form a data word of the first set of data words;
if the value is a numerical value, generating at least one description of the value, and appending each of the at least one description to the prefix to form at least one data word of the first set of data words.

14. The method of claim 13, wherein generating the at least one description of the value comprises calculating a mean and a standard deviation of a set of values having the same prefix and wherein the at least one description of the value is based on the value, the mean, and the standard deviation.

15. The method of claim 10, wherein the first combined text includes medical data and wherein the predictive model built by the predictive modeling engine, when executed, predicts a set of medical codes.

16. A computer-implemented method for classifying data using the predictive model built by the method of claim 10, wherein the predictive model predicts membership in a set of classes, the method comprising:
receiving, by at least one processor, second structured data;
generating, by the at least one processor from the second structured data, a second set of data words, wherein each data word in the second set of data words has a prefix and a value;
combining, by the at least one processor, the second set of data words to form second combined text, wherein the second combined text is free text; and
executing, by the at least one processor, the predictive model to classify the second combined text into at least one class of the set of classes.

17. The method of claim 16, wherein each data word of the first set of data words is followed by a separator and wherein the classifying comprises generating, by the at least one processor, an explanation for the classification made by the predictive model.

18. A computer-implemented method for classifying free text using the predictive model built by the method of claim 11, the method comprising:
receiving, by at least one processor, second free text and second structured data, wherein the second structured data corresponds to data in the second free text;
generating, by the at least one processor, from the second structured data, a second set of data words, wherein each data word in the second set of data words has a prefix and a value;
combining, by the at least one processor, the second set of data words into second combined text, wherein the second combined text is free text; and
executing, by the at least one processor, the predictive model to classify the second combined text into at least one class of the set of classes.

19. The method of claim 18, wherein the at least one processor generates the second structured data from the second free text.

20. The method of claim 18, wherein each data word of the second set of data words is followed by a separator and wherein the classifying comprises generating, by the at least one processor, an explanation for the classification made by the predictive model.
